(19) United States
(12) Patent Application Publication
(10) Pub. No.: US 2022/0218297 A1
(43) Pub. Date: Jul. 14, 2022

(54) MOBILE RADIOGRAPHY SYSTEM, RECORDING MEDIUM, AND RADIOGRAPHY SCHEDULE GENERATION METHOD

(71) Applicant: Konica Minolta, Inc., Tokyo (JP)

(72) Inventors: Sumiya NAGATSUKA, Tokyo (JP); Ikki NAKAMURA, Tokyo (JP)

(21) Appl. No.: 17/573,770

(22) Filed: Jan. 12, 2022

(30) Foreign Application Priority Data
Jan. 12, 2021 (JP) 2021-002508

(51) Int. Cl. A61B 6/00

(52) U.S. Cl. CPC A61B 6/4405; A61B 6/56; A61B 6/4494

(57) ABSTRACT

A mobile radiography system generates a radiography schedule of dynamic radiography through use of radiation, and performs the dynamic radiography. The system includes an acquisition unit, a generator, and an output unit. The acquisition unit acquires order information about each human subject concerning the dynamic radiography. The generator generates the radiography schedule of the dynamic radiography including a pause process based on the order information. The output unit outputs the radiography schedule.

### CROSS REFERENCES TO RELATED APPLICATIONS

The present invention claims priority under 35 U.S.C. § 119 to Japanese Application, 2021-002508, filed on Jan. 12, 2021, the entire contents of which being incorporated herein by reference.

### BACKGROUND

### 1. Technological Field

The present invention relates to a mobile radiography system, a recording medium, and a radiography schedule generation method.

### 2. Description of the Related Art

A conventionally known mobile radiography device has an object to perform radiography in a traveling diagnosis in a hospital. Various technologies for radiography management for facilitating a traveling diagnosis involving radiography through use of a mobile radiography device have been proposed.

JP 2017-99783A, for example, describes deriving and displaying a radiography order that prevents remaining battery power of a flat panel detector (FPD) to be used in a mobile radiography device from becoming insufficient. JP 2017-99783A also describes displaying to the effect that it is time to charge if a radiography order that prevents remaining battery power from becoming insufficient is not derived and remaining battery power of the FPD becomes insufficient during a traveling diagnosis.

JP 2006-340788A and JP 2018-158026A, for example, describe systems that perform information management concerning traveling diagnosis schedules and changes in the traveling diagnosis schedules for traveling diagnoses through use of a plurality of mobile radiography devices.

However, it has been found that dynamic radiography through use of a mobile radiography device involves various variable factors, such as the remaining battery power of the mobile radiography device, heat generation of a radiation source, and a remaining memory capacity of the FPD, in addition to the battery of the FPD, and such a variable factor may make it impossible to continue mobile radiography during a traveling diagnosis. JP 2017-99783A discloses deriving a radiography order that prevents remaining battery power of the FPD from becoming insufficient, but fails to mention deriving a radiography schedule which considers the possibility that a variable factor other than insufficiency of remaining battery power of the FPD makes it impossible to continue mobile radiography. Thus, a radiographer, nurse, or the like, for example, suddenly becomes aware that mobile radiography can no longer be continued during a traveling diagnosis. The radiographer, nurse, or the like becomes unable to conduct work in a scheduled manner, resulting in inefficient work. JP 2006-340788A and JP 2018-158026A fail to mention managing a traveling diagnosis schedule of dynamic radiography.

### SUMMARY

The present invention was made in view of the above problems, and has an object to provide an efficient radiography schedule for a traveling diagnosis that performs dynamic radiography considering various variable factors specific to dynamic.

To achieve the object, according to an aspect of the present invention, a mobile radiography system that generates a radiography schedule of dynamic radiography through use of radiation and performs the dynamic radiography includes:

an acquisition unit that acquires order information about each human subject concerning the dynamic radiography;

a generator that generates the radiography schedule of the dynamic radiography including a pause process based on the order information; and

an output unit that outputs the radiography schedule.

According to another aspect of the present invention, a non-transitory computer-readable recording medium stores a computer-readable program that causes a computer that generates a radiography schedule of dynamic radiography through use of radiation to function as:

an acquisition unit that acquires order information about each human subject concerning the dynamic radiography;

a generator that generates the radiography schedule of the dynamic radiography including a pause process based on the order information; and

an output unit that outputs the radiography schedule.

According to still another aspect of the present invention, a radiography schedule generation method of generating a radiography schedule of dynamic radiography through use of radiation includes:

acquiring order information about each human subject concerning the dynamic radiography;

generating the radiography schedule of the dynamic radiography including a pause process based on the order information; and

outputting the radiography schedule.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a diagram showing an overall configuration example of a mobile radiography system.

FIG. 2 is a block diagram showing a functional configuration of the mobile radiography system of FIG. 1.

FIG. 3 is a flow chart showing radiography schedule generation processing executed by a controller of FIG. 2.

FIG. 4 is a diagram showing an example of a radiography schedule display screen.

FIG. 5 is a flow chart showing traveling diagnosis processing executed by the controller of FIG. 2.

FIG. 6 is a diagram showing an example of a details screen.

### DETAILED DESCRIPTION OF THE EMBODIMENTS

Hereinafter, an embodiment of the present invention will be described with reference to the drawings. However, the scope of the invention is not limited to the illustrated examples.

Configuration of Mobile Radiography System 100

First, a configuration of an embodiment of the present invention will be described.

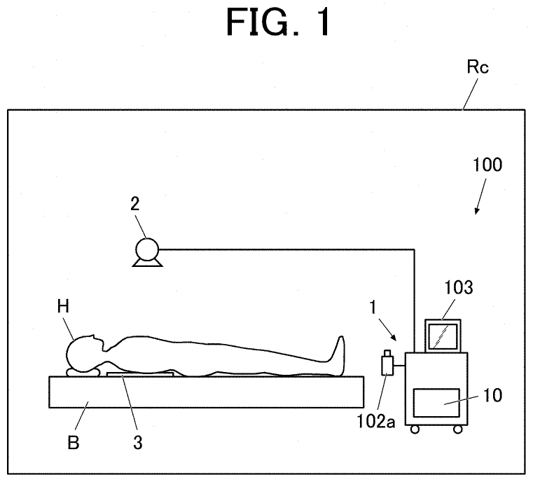

FIG. 1 shows an overall configuration example of a mobile radiography system 100 of the present embodiment.

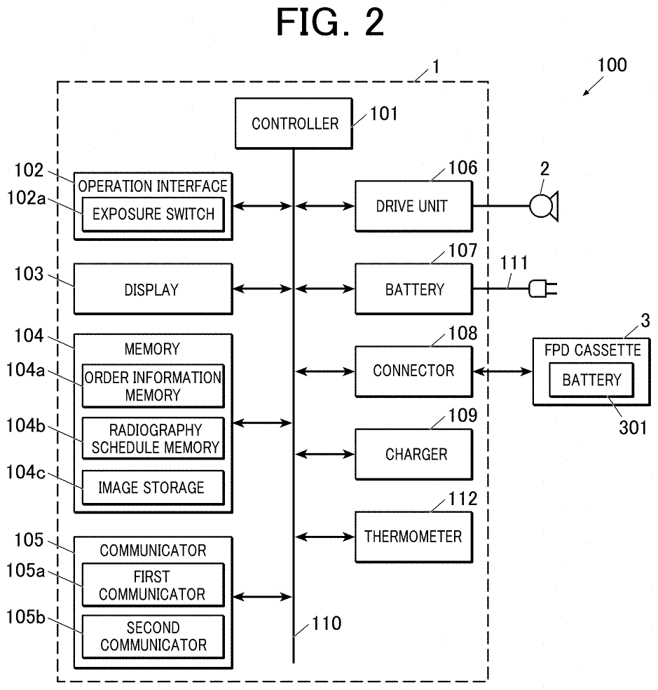

The mobile radiography system 100 is, for example, a system for radiographing a patient difficult to move in a traveling diagnosis, and has a function of generating a radiography schedule of mobile radiography and a function of executing dynamic radiography. Herein, the dynamic radiography is radiography for radiographing a moving state or a varying state, and recording video. The dynamic radiography includes video radiography. The dynamic radiography does not include radiographing a still image while displaying video. The mobile radiography system 100 includes a main body 1, a radiation source 2, and an FPD cassette 3. The mobile radiography system 100 is implemented as a movable medical vehicle having wheels on the main body 1. The main body 1 is provided with a housing 10 for housing the FPD cassette 3. The housing 10 is provided with a connector 108 (see FIG. 2) for connection with the FPD cassette 3 being housed, and is conveyable while charging a battery 301 (see FIG. 2) of the FPD cassette 3 being housed.

In the present embodiment, the mobile radiography system 100 will be described using, as an example, a case in which the main body 1 implemented as a medical vehicle has the function of generating a radiography schedule, however, the function of generating a radiography schedule may be provided for a device (such as a personal computer (PC), for example) separate from the medical vehicle. The mobile radiography system 100 may be a portable system having no wheels.

As shown in FIG. 1, the mobile radiography system 100 is brought into a surgery room, an intensive care unit, a room Rc, or the like, and emits radiation from the radiation source 2 with the FPD cassette 3 being inserted between a subject H (human subject) lying on a bed B and the bed B or inserted into an insertion slot not shown but provided in a surface of the bed B opposite to the subject H, for example, thereby performing still radiography or dynamic radiography for the subject H. In the present embodiment, the still radiography refers to acquiring an image of the subject in accordance with a single radiography operation (pressing of an exposure switch 102a). The dynamic radiography refers to acquiring a plurality of images presenting dynamic of a subject by repeatedly irradiating the subject with radiation such as X rays in a pulsed manner at predetermined time intervals (pulsed radiation) or continually irradiating the subject with radiation without interruption at a low dose rate (continuous radiation). A series of images obtained by the dynamic radiography are called a dynamic image. Each of the plurality of images constituting the dynamic image is called a frame image.

FIG. 2 is a block diagram showing a functional configuration of the mobile radiography system 100.

As shown in FIG. 2, the main body 1 of the mobile radiography system 100 includes a controller 101, an operation interface 102, a display 103, a memory 104, a communicator 105, a drive unit 106, a battery 107, a connector 108, a charger 109, a thermometer 112, and the like, which are connected to each other with a bus 110.

The controller 101 includes a central processing unit (CPU), a random access memory (RAM), and the like. The CPU of the controller 101 reads out a system program and various processing programs stored in the memory 104 in accordance with input through the operation interface 102, expands them into the RAM, and executes various types of processing including radiography schedule generation processing which will be described later in accordance with the expanded programs. The controller 101 functions as an acquisition unit and a generator.

The operation interface 102 has a touch panel or the like in which transparent electrodes are arranged in a lattice so as to cover the surface of the display 103, and detects a position pressed by a finger, a touch pen, or the like and inputs positional information thereof to the controller 101 as operation information.

The operation interface 102 has the exposure switch 102a for a user to instruct start of radiation exposure.

The display 103 is implemented by a monitor such as a liquid crystal display (LCD) or a cathode ray tube (CRT), and provides display in accordance with an instruction of a display signal input from the controller 101. The display 103 functions as an output unit.

The memory 104 is implemented by a nonvolatile semiconductor memory, a hard disk, or the like. The memory 104 stores various programs to be executed by the controller 101, parameters necessary for executing processing in accordance with the programs, or data such as processing results.

For example, the memory 104 is provided with an order information memory 104a, a radiography schedule memory 104b, an image storage 104c, and the like.

The order information memory 104a stores order information received from a radiology information system (RIS) not shown. Herein, the order information is radiography-related information about each human subject, and includes, for example, examination identification information (such as an examination ID), an examination date, information concerning a human subject (patient information (such as the name, the sex, the age, a physical constitution (such as height and weight), and a room), information concerning radiography to be executed for the human subject (a radiography ID, a diagnostic purpose, a radiography type indicating still radiography or dynamic radiography, radiography conditions (such as, for example, a radiography part, a radiography direction, a frame rate, the number of radiographs, radiation power of the radiation source 2 (such as a tube voltage, a tube current, and a radiation time)), and notes (such as, for example, radiography in a surgery room, emergency, and priority)). The patient information may include present conditions of the human subject (such as breathing difficulty), a disease, a level of care needed, and the like. The controller 101 may derive the radiography conditions based on the physical constitution and the like included in the order information.

The radiography schedule memory 104b stores information about a radiography schedule generated by radiography schedule generation processing which will be described later.

The image storage 104c is a memory region that temporarily stores image data acquired by mobile radiography before transfer to an external device (such as an analysis device or a picture archiving and communication system (PACS)).

The memory 104 also stores information about heat capacity of the radiation source 2, information about the temperature of heat generated by a single standard dynamic radiography session, capacities of the battery 107 and the battery 301, a storage capacity (memory capacity) of the image storage 104c, a hospital map, a traveling diagnosis route, a traveling diagnosis start time and finish time, information concerning traveling diagnosis targets per day of week, a radiography priority, various tables, and the like. The hospital map includes, for example, information such as positions of rooms, surgery rooms, emergency rooms, and the like in a hospital, positions of elevators and the like where radio waves do not reach or are weak, and a communication speed at each spot in the hospital, and the like.

The communicator 105 includes a first communicator 105a for transmitting/receiving data to/from the FPD cassette 3, and a second communicator 105b for transmitting/receiving data by wireless communication to/from an external device such as an RIS, an analysis device, or a PACS connected to a communication network (in-hospital network) such as a local area network (LAN) or a wide area network (WAN) via wireless access points provided at various places in the hospital.

The drive unit 106 is a circuit that drives a tube of the radiation source 2. The drive unit 106 and the radiation source 2 are connected to each other via a cable.

The battery (power storage) 107 supplies power to each part of the main body 1 and the radiation source 2. The battery 107 is externally rechargeable via an AC cable 111. The battery 107 is previously charged via the AC cable 111 in a time slot in which there is no radiography work, and the AC cable 111 is housed within the main body 1 during movement.

The connector 108 is provided inside the housing 10, and electrically connects to the FPD cassette 3 housed in the housing 10.

The charger 109 is a circuit that charges the battery 301 of the FPD cassette 3 connected via the connector 108 by power supplied from the battery 107 while radiography is not being performed, based on control exerted by the controller 101.

The thermometer 112 is provided in the vicinity of the radiation source 2, and includes a thermometer that measures the temperature of the radiation source 2 and a thermometer that measures the room temperature of a room in which the mobile radiography system 100 is provided.

The radiation source 2 is driven by the drive unit 106, and irradiates the subject H with radiation (X rays).

The FPD cassette 3 is a radiography device that generates a radiation image (image data) based on radiation emitted from the radiation source 2. The FPD cassette 3 is a portable radiation detector in which the rechargeable battery (power storage) 301 serves as a drive source, and is adaptable to still radiography and dynamic radiography. The FPD cassette 3 has a glass substrate and the like, for example, and a plurality of detection elements are arrayed two-dimensionally at a predetermined position on the substrate, the detection elements detecting radiation emitted from the radiation source 2 and at least passed through the subject H in accordance with the intensity of the radiation, converting the detected radiation into an electric signal, and accumulating the electric signals. The detection element is implemented by a semiconductor image sensor such as a photodiode. Each detection element is connected to a switching unit such as a thin film transistor (TFT), for example. Accumulation and read-out of an electric signal is controlled by the switching unit, and image data (a frame image) is acquired.

The FPD includes an indirect conversion type FPD that converts radiation into an electric signal by a photoelectric conversion element via a scintillator and a direct conversion type FPD that directly converts radiation into an electric signal, and either may be used as the FPD cassette 3.

Operation of Mobile Radiography System 100

Next, operation of the mobile radiography system 100 will be described.

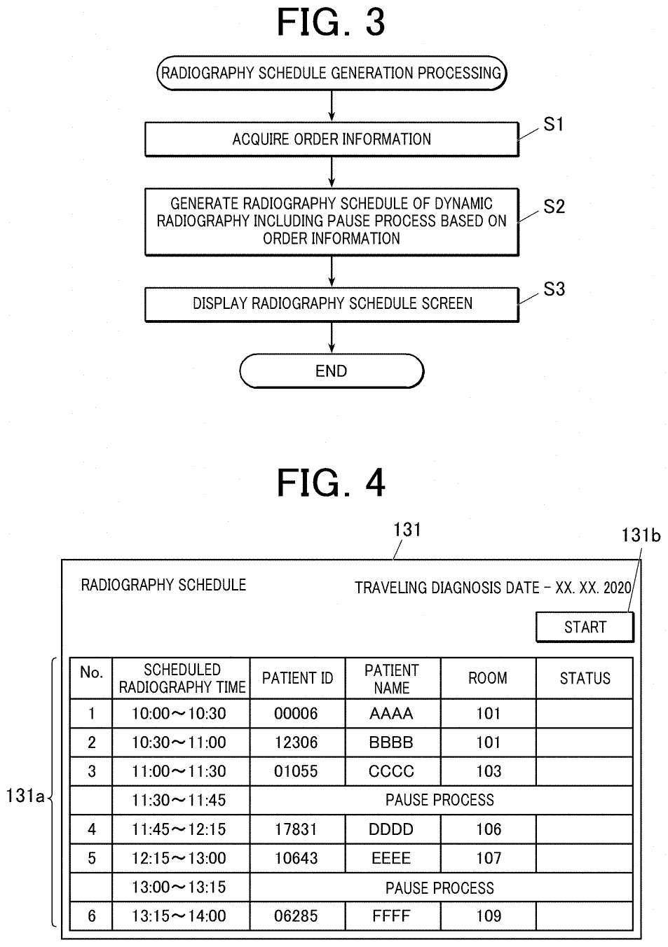

FIG. 3 is a flow chart showing a flow of radiography schedule generation processing executed by the controller 101. When an instruction for generating a radiography schedule of dynamic radiography in a traveling diagnosis is input through the operation interface 102, the controller 101 and a program stored in the memory 104 cooperate to execute the radiography schedule generation processing. The present embodiment will be described using, as an example, a case of performing dynamic radiography in a traveling diagnosis.

First, the controller 101 acquires, from a RIS, order information about dynamic radiography to be executed in a traveling diagnosis (Step S1).

For example, the controller 101 connects to the RIS, which is not shown, via the communicator 105, acquires, from the RIS, a list of order information about dynamic radiography to be executed in a traveling diagnosis, and causes the display 103 to display the list and causes the order information memory 104a to store the list.

The controller 101 then generates a radiography schedule of dynamic radiography including a pause process based on the acquired order information (Step S2).

In Step S2, the controller 101 first determines a radiography order based on the acquired order information, for example.

In a medical facility, for example, a traveling diagnosis route indicating from which room a traveling diagnosis is to be performed successively, a traveling diagnosis start time, a traveling diagnosis finish time, the day of week on which a traveling diagnosis targeted for emergent patients is to be performed, and the like are determined previously. The memory 104 stores such information concerning the traveling diagnosis route, the traveling diagnosis start time and finish time, diagnosis targets per day of week, a hospital map, and the like. The controller 101 refers to these pieces of information stored in the memory 104, and determines the radiography order based on the acquired order information. The traveling diagnosis route, the traveling diagnosis start time and finish time, and the diagnosis targets may be input by a user operating the operation interface 102.

The controller 101 then interposes a pause process between radiography sessions. The dynamic radiography involves radiographing many images at a time. It is therefore assumed that, when dynamic radiography is performed in a traveling diagnosis, variations in device state, such as heat generation of the radiation source 2, insufficient remaining power of the battery 107 or the battery 301, and insufficient remaining capacity (insufficient remaining memory capacity) of the image storage 104c, may occur during the traveling diagnosis, and may make it impossible to continue mobile radiography. In the case where a radiographer or nurse suddenly becomes aware that the mobile radiography can no longer be continued during the traveling diagnosis, the radiographer or nurse becomes unable to do work in a scheduled manner, resulting in inefficient work. The present embodiment therefore sets a pause process between radiography sessions in the radiography schedule. The pause process is a process of pausing dynamic radiography for dissipating heat of the radiation source 2, for charging the power storage (the battery 107 or the battery 301), or for transferring image data to ensure a free space in the image storage 104c (free up the memory).

For example, the controller 101 interposes a pause process between dynamic radiography for a first human subject and dynamic radiography for a second human subject (that is, between radiography for a certain human subject and radiography for the next human subject). Alternatively, a pause process may be interposed between dynamic radiography in a first room and dynamic radiography in a second room (that is, at the transition to radiography in a different room).

Between which radiography sessions a pause process is to be interposed specifically, and the length of each pause process are determined based on, for example, at least one of:

information concerning heat generation of the radiation source;

information about power storage concerning mobile radiography;

information about a memory that stores a dynamic image obtained by dynamic radiography; and

information about wireless communication.

The length of the pause process may further be determined based on information concerning a use environment of the mobile radiography system 100.

The information concerning heat generation of the radiation source includes at least one of:

information about the heat capacity of the radiation source 2; and

information about the temperature of heat generated by a single standard dynamic radiography session.

The information about power storage concerning mobile radiography includes information of at least one of:

the capacity and the present amount of stored power of the battery 301 of the FPD cassette 3; and

the capacity and the present amount of stored power of the battery 107 which is the power storage of the main body 1.

The capacity and the present amount of stored power of the battery 301 of the FPD cassette 3 may be:

a value of the capacity and a value of the present amount of stored power; or a value representing the ratio between the present amount of stored power and the capacity of the battery 301.

The capacity and the present amount of stored power of the battery 107 which is the power storage of the main body 1 may be:

a value of the capacity and a value of the present amount of stored power; or

a value representing the ratio between the present amount of stored power and the capacity of the battery 107 which is the power storage of the main body 1.

The information about the memory that stores a dynamic image obtained by dynamic radiography includes information about the storage capacity and the present remaining capacity (free space) of the image storage 104c.

The storage capacity and the present remaining capacity (free space) of the image storage 104c may be:

a value of the storage capacity and a value of the present remaining capacity; or

a value representing the ratio between the present remaining capacity and the storage capacity.

The information about wireless communication includes:

information indicating the positions of elevators and the like where radio waves do not reach or are weak (information indicating a second position at which radio waves are weaker than at a first position in the hospital); or

information about the communication speed at each spot in the hospital.

The information concerning the use environment of the mobile radiography system 100 includes at least one of a room temperature and a network communication band (communication speed).

For example, in the case of interposing a pause process between dynamic radiography for the first human subject and dynamic radiography for the second human subject (that is, between radiography for a certain human subject and radiography for the next human subject), to what degree the temperature of the radiation source 2 is raised by radiography for the first human subject is predicted based on:

information about the heat capacity (for example, a heat unit (HU) value) of the radiation source 2; and

the amount of heat of the radiation source 2 predicted based on:
the present temperature of the radiation source 2 (in a case where radiography for the first human subject is not initial radiography, the temperature of the radiation source 2 at the start of radiography for the first human subject (information about the temperature of the radiation source 2 after immediately preceding radiography or after the pause process)); and
radiography conditions (such as, for example, a tube current, a tube voltage, and a radiation time) included in the order information about the first human subject, for example.

In accordance with the predicted temperature of the radiation source 2, whether to interpose a pause process, and in a case of interposing a pause process, what length the pause process has, are determined. To what temperature the radiation source 2 is raised by radiography for the first human subject may be predicted based on the information about the temperature of heat generated by a single standard dynamic radiography session. For determining the length of the pause process, a table of the temperature of the radiation source 2 and the length of the pause process is previously stored in the memory 104, and the controller 101 refers to the table to determine the length of the pause process. Because the cooling speed of the radiation source 2 varies according to the room temperature, a table of the temperature of the radiation source 2 and the length of the pause process may be prepared for each room temperature, and the length of the pause process may be determined taking the present room temperature into consideration.

The remaining power (the amount of stored power) of the battery 107 after radiography for the first human subject is calculated based on, for example:

information about the present amount of stored power of the battery 107 (in the case where radiography for the first human subject is not initial radiography, information about the amount of stored power at the start of radiography for the first human subject (information about the amount of stored power after immediately preceding radiography or after a pause process)); and

the amount of power consumption of the battery 107 predicted based on radiography conditions (such as, for example, the number of radiographs, a tube current, a tube voltage, and a radiation time) included in the order information about the first human subject.

Based on the calculated remaining power of the battery 107, whether to interpose a pause process after radiography, and in the case of interposing a pause process, what length the pause process has, are determined. For example, the amount of charge of the battery 107 per unit time is stored in the memory 104, and the length of the pause process is determined based on the amount of charge of the battery 107 per unit time, the capacity (or a previously determined target capacity) of the battery 107, and the remaining power after radiography.

The remaining power (the amount of stored power) of the battery 301 after radiography for the first human subject is calculated based on, for example:

information about the present amount of stored power of the battery 301 (in the case where radiography for the first human subject is not initial radiography, information about the amount of stored power at the start of radiography for the first human subject (information about the amount of stored power after immediately preceding radiography or after a pause process)); and

the amount of power consumption of the FPD cassette 3 predicted based on radiography conditions (such as, for example, the number of radiographs, a tube current, a tube voltage, and a radiation time) included in the order information about the first human subject.

Based on the calculated remaining power of the battery 301, whether to interpose a pause process, and in the case of interposing a pause process, what length the pause process has, are determined.

The amount of charge of the battery 301 per unit time is stored in the memory 104, and the length of the pause process is determined based on, for example:

the amount of charge of the battery 301 per unit time; and

a difference between the capacity (or a previously determined target capacity) of the battery 301 and the remaining power after radiography.

The remaining capacity (free space) of the image storage 104c after radiography for the first human subject is calculated based on, for example:

information about the present remaining capacity of the image storage 104c (in the case where radiography for the first human subject is not initial radiography, information about the remaining capacity at the start of radiography for the first human subject (information about the remaining capacity after immediately preceding radiography or after a pause process)); and

the amount of image data predicted based on radiography conditions (such as, for example, the number of radiographs and the number of pixels of the FPD cassette 3 used) included in the order information about the first human subject.

Based on the calculated remaining capacity of the image storage 104c, whether to interpose a pause process after radiography is determined.

In the case of interposing a pause process, what length the pause process has is determined based on, for example:

usage of the image storage 104c, calculated based on the capacity of the image storage 104c and the remaining capacity of the image storage 104c after radiography; and

information about wireless communication (for example, a wireless communication speed) at a place where the pause process is executed.

Information obtained by a single standard dynamic radiography session may simply be stored in the memory 104 previously, and the amount of heat of the radiation source 2 generated by radiography, the amount of power consumption of the batteries 107 and 301, and the amount of image data may be predicted using the stored information.

The amount of power consumption, the amount of heat, and the amount of image data may be predicted considering re-radiography. For example, the number of re-radiography sessions for at least one of each disease, each level of care needed, each age, and each condition may be obtained statistically and stored in the memory 104. The number of re-radiography sessions may be predicted based on the disease, the level of care needed, the age, or conditions of a human subject. The amount of power consumption, the amount of heat, and the amount of image data may be predicted considering re-radiography of the predicted number of re-radiography sessions.

The length of a pause process which is the longest among the pause processes respectively determined for the radiation source 2, the battery 107, the battery 301, and the image storage 104c, for example, is determined as the length of the pause process to be interposed between dynamic radiography for the first human subject and dynamic radiography for the second human subject.

In the case of interposing a pause process between dynamic radiography in the first room and dynamic radiography in the second room, the amount of heat of the radiation source 2, the amount of power consumption of the battery 107, the amount of power consumption of the battery 301, and the amount of image data in the whole radiography executed in the first room are predicted, while the amount of heat of the radiation source 2, the amount of power consumption of the battery 107, the amount of power consumption of the battery 301, and the amount of image data in the radiography for the first human subject are predicted in the pause process determination technique in the above-described case of interposing a pause process between dynamic radiography for the first human subject and dynamic radiography for the second human subject.

The controller 101 then predicts a time required for each radiography session and a travel time.

The time required for each radiography session is the sum of a radiography preparation time and a radiography time, for example. The radiography time is predicted based on, for example, radiography conditions (such as a frame rate, the number of radiographs, and radiation power of the radiation source) in the order information. The number of re-radiography sessions may be predicted based on at least one of the disease, the level of care needed, the age, and conditions of a human subject, and the radiography time including the re-radiography may be predicted. The radiography preparation time may be uniform, but it is assumed that the time required for radiography preparation varies according to the human subject's state in such a way that, for example, a longer time is required for radiography preparation for an immobile human subject, or a longer time is required for a human subject having a large physical constitution (for example, tall or heavy). It is therefore preferable to predict the radiography preparation time based on at least one of the disease, the level of care needed, the age, the conditions, and the physical constitution of the human subject. Because a longer time is required for radiography preparation as a smaller number of radiographers and nurses (staff) perform a traveling diagnosis, the number of staff members who perform the traveling diagnosis may previously be set, and the radiography preparation time may be predicted considering the number of staff members. These predictions are performed by, for example, storing, in the memory 104, a table of the radiography preparation time for each human subject's state and/or each number of staff members based on statistics on the radiography preparation time for each human subject's state and/or each number of staff members in radiography sessions executed in the past, or the like and referring to the table.

The travel time is set between radiography sessions in a case of moving to a different room and executing the next radiography, for example, and is predicted based on, for example, the distance between spots on the hospital map, or the like. In a case where both the travel time and the pause process are interposed between radiography sessions, a longer one of the travel time and the pause time may be adopted and integrated.

The controller 101 then determines a scheduled start time and scheduled finish time of each radiography session, and a scheduled start time and scheduled finish time of a pause process based on the traveling diagnosis start time, the radiography order including pause processes, the time required for each radiography session, the travel time, and the length of each pause process, and generates a radiography schedule. The controller 101 then causes the radiography schedule memory 104b to store information about the generated radiography schedule (radiography schedule information). The radiography schedule information preferably includes predicted information about the temperature of the radiation source 2, the remaining power (the amounts of stored power) of the battery 107 and the battery 301, and the remaining capacity (free space) of the image storage 104c after the end of each radiography session calculated in the process of generating the radiography schedule, and shall include these types of information in the present embodiment.

The controller 101 then causes the display 103 to display a radiography schedule screen 131 on which the generated radiography schedule is displayed (Step S3), and finishes the radiography schedule generation processing.

FIG. 4 is a diagram showing an example of the radiography schedule screen 131 displayed on the display 103. As shown in FIG. 4, a radiography schedule 131a having been generated and a start button 131b for instructing the start of a traveling diagnosis are displayed on the radiography schedule screen 131. A radiographer or nurse who performs the traveling diagnosis checks the radiography schedule 131a, and presses the start button 131b to start the traveling diagnosis.

Herein, a plurality of radiography schedules may be generated. For example, the controller 101 generates a first radiography schedule including a pause process (called a first pause process) having a length determined by the technique described with reference to Step S2 or the like and a second radiography schedule including a larger number of pause processes than in the first radiography schedule, the pause processes each being shorter than the first pause process. When displaying the radiography schedules in Step S3, the first radiography schedule and the second radiography schedule may be displayed on the display 103 at the same time, and one of the radiography schedules selected by a user through the operation interface 102 may be displayed on the radiography schedule screen 131 to be employed for a traveling diagnosis.

Alternatively, the above-described first radiography schedule may be generated, and the radiography schedule screen 131 on which the first radiography schedule is displayed may be displayed on the display 103. In such a case where the first radiography schedule does not fit user's needs, the second radiography schedule may be generated in accordance with an operation of pressing a “different schedule” button (not shown) provided on the radiography schedule screen 131, and the radiography schedule screen 131 on which the second radiography schedule is displayed may be displayed on the display 103.

A third radiography schedule not including a pause process may also be generated. For example, the first radiography schedule and the second radiography schedule are generated so as to include dynamic radiography for all human subjects that should be executed in a traveling diagnosis on the day. Further, the third radiography schedule including radiography only for emergency patients or only for high-priority patients and not including a pause process is generated. Then, the first to third radiography schedules may be displayed on the display 103 at the same time, and one of the radiography schedules selected by the user through the operation interface 102 may be displayed on the radiography schedule screen 131 to be employed for the traveling diagnosis. Alternatively, the third radiography schedule may be generated without generating the first radiography schedule and the second radiography schedule in accordance with user's needs, that is, in accordance with an instruction through the operation interface 102, and may be displayed on the radiography schedule screen 131 to be employed for the traveling diagnosis.

This enables a radiography schedule that fits the user's needs to be generated and employed for the traveling diagnosis.

When the start button 131b is pressed through the operation interface 102 on the radiography schedule screen 131, and the start of mobile radiography is instructed, the controller 101 executes the traveling diagnosis processing.

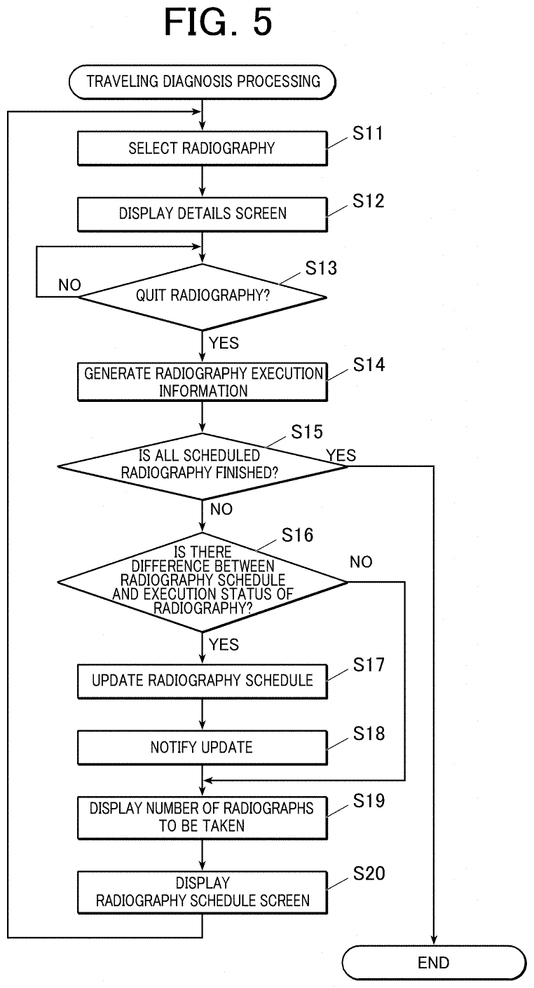

FIG. 5 is a flow chart showing a flow of the traveling diagnosis processing executed by the controller 101 when the start button 131b is pressed. The traveling diagnosis processing is executed by cooperation of the controller 101 and a program stored in the memory 104.

In the traveling diagnosis processing, the controller 101 first accepts an operation through the operation interface 102 of selecting radiography to be executed next from the radiography schedule 131a displayed on the radiography schedule screen 131 (Step S11).

When radiography to be executed next is selected, the controller 101 causes the display 103 to display a details screen 132 (Step S12).

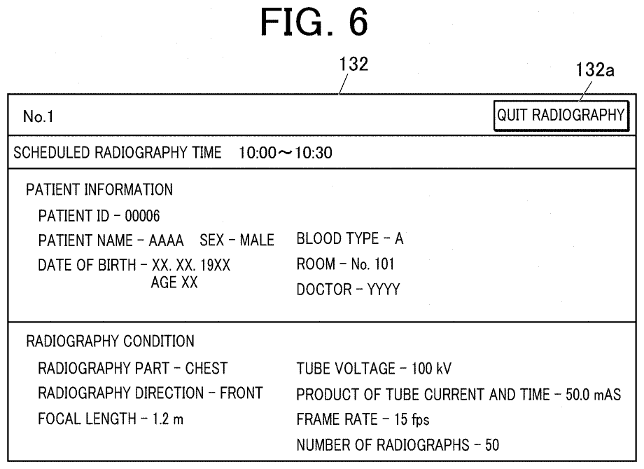

FIG. 6 is a diagram showing an example of the details screen 132. As shown in FIG. 6, details concerning the selected radiography, such as a scheduled radiography time of the selected radiography, patient information, and radiography conditions, are displayed on the details screen 132. A staff member who performs a traveling diagnosis sets and checks the radiography conditions while watching the details screen 132. A radiography quit button 132a is displayed on the details screen 132.

The controller 101 then waits for pressing of the radiography quit button 132a through the operation interface 102 (Step S13).

When the radiography quit button 132a is pressed (YES in Step S13), the controller 101 generates radiography execution information about the radiography schedule for which radiography is finished, and causes the order information memory 104a to store the generated radiography execution information in association with information about the radiography schedule (Step S14).

The radiography execution information preferably includes, for example, information about the radiography finish time, the temperature of the radiation source 2 after radiography, the remaining power (the amounts of stored power) of the battery 107 and the battery 301, and the remaining capacity (free space) of the image storage 104c, and shall include these types of information in the present embodiment.

The controller 101 then determines whether all scheduled radiography is finished based on the radiography schedule information and the radiography execution information (Step S15).

In a case where it is determined that all scheduled radiography is not finished (NO in Step S15), the controller 101 determines whether there is a difference between the radiography schedule and an execution status of radiography (progress status) based on the radiography schedule information and the radiography execution information (Step S16).

For example, when a longer time is taken for radiography preparation or when the number of re-radiography sessions is larger than predicted when scheduling radiography, the radiography finish time is delayed, or the states of the mobile radiography system 100, such as the temperature of the radiation source 2 after radiography, the remaining power (the amounts of stored power) of the battery 107 and the battery 301, and the remaining capacity (free space) of the image storage 104c, no longer agree with those when radiography is scheduled.

Thus, pieces of information about these items in the radiography schedule information and the radiography execution information are compared to determine whether there is a difference between the radiography schedule and the execution status of radiography.

In a case where it is determined that there is no difference between the radiography schedule and the execution status of radiography (NO in Step S16), the controller 101 causes the display 103 to display the number of radiographs to be taken (Step S19), and then causes the screen of the display 103 to transition to the radiography schedule screen 131 (Step S20), and returns to Step S11.

In a case where it is determined that there is a difference between the radiography schedule and the execution status of radiography (progress status) (YES in Step S16), the controller 101 updates the radiography schedule (Step S17).

In Step S17, the controller 101 updates the radiography schedule based on information about an item for which there is a difference.

For example, in a case where an initially generated radiography schedule schedules that the usage of the image storage 104c up to the present time point is 60% (remaining capacity: 40%), but re-radiography occurs more than initially scheduled and the usage is 80% (remaining capacity: 20%), the controller 101 determines whether image data to be acquired before the next pause process can be stored with that remaining capacity, and in a case where it is determined that the image data cannot be stored, updates the radiography schedule so as to move up the position of the pause process.

Likewise, for example, in a case where the initially generated radiography schedule schedules that the temperature of the radiation source 2 has a margin of 20% with respect to a predetermined temperature at the present time point, but re-radiography occurs more than initially scheduled, resulting in a mere 5% margin, the controller 101 determines whether the radiation source 2 exceeds the predetermined temperature before the next pause process, and in a case where the radiation source 2 exceeds the predetermined temperature, updates the radiography schedule so as to move up the position of the pause process.

Likewise, for example, in a case where the initially generated radiography schedule schedules that the remaining power (the amount of stored power) of the battery 107 or 301 is 60% at the present time point, but re-radiography occurs more than initially scheduled, resulting in the remaining power of 20%, the controller 101 determines whether battery exhaustion occurs before the next pause process, and in a case where it is determined that battery exhaustion occurs, updates the radiography schedule so as to move up the position of the pause process.

In a case where there is a difference (delay) of more than or equal to a predetermined time in the radiography finish time as compared with the initially generated radiography schedule, the controller 101 determines that radiography according to the radiography schedule can no longer be performed completely until the end, and displays on the display 103 a warning that radiography according to the radiography schedule can no longer be finished completely until the end. The warning may be output by a voice, a buzzer, or the like. The controller 101 then refers to the memory 104, and updates the radiography schedule based on a previously set radiography priority.

For example, in a case where “radiography for all scheduled human subjects” is given the highest priority, the radiography conditions of remaining radiography are changed, and the radiography schedule is updated. Examples of changing of the radiography conditions include changing remaining dynamic radiography to still radiography. Preferably, radiography that may be changed to still radiography is previously set.

For example, in a case where “complete radiography for a high-priority human subject” is given the highest priority, radiography to be executed is reduced, and the radiography schedule is updated. For radiography yet to be executed, for example, the radiography schedule is changed and updated to execute radiography only for a high-priority human subject or an emergent human subject. Alternatively, for radiography yet to be executed, the radiography schedule may be changed to first execute radiography for a high-priority human subject or an emergent human subject, and execute radiography for other human subjects for the remaining time until the traveling diagnosis finish time.

When changing the radiography conditions or radiography to be executed, the start position and length of the pause process are also changed according to necessity.

The controller 101 then causes the display 103 to display a radiography schedule update notification (Step S18).

The controller 101 then causes the display 103 to display the number of radiographs to be taken (Step S19), causes the screen of the display 103 to transition to the radiography schedule screen 131 (Step S20), and returns to Step S11.

In a case where it is determined in Step S15 that all scheduled radiography is finished (YES in Step S15), the controller 101 finishes the traveling diagnosis processing.

Modification

The above-described embodiment has been described using, as an example, a case in which the controller 101 generates a radiography schedule only for dynamic radiography, but the controller 101 may have a function of generating a radiography schedule in which dynamic radiography and still radiography are mixed, and may further have a function of generating a radiography schedule only for still radiography. The radiography schedule in which dynamic radiography and still radiography are mixed and the radiography schedule only for still radiography can also be generated by a technique similar to that described with reference to Step S2 of FIG. 3.

As described above, according to the mobile radiography system 100, the controller 101 acquires order information concerning dynamic radiography, and generates a radiography schedule of dynamic radiography including a pause process based on the acquired order information. The controller 101 then displays (outputs) the generated radiography schedule by the display 103.

The radiography schedule is thereby generated considering that various variable factors, such as the remaining power (the amount of stored power) of the battery 107 or 301, heat generation of the radiation source 2, and the remaining capacity (free space) of the image storage 104c that stores dynamic images, may make it impossible to continue mobile radiography during a traveling diagnosis. This prevents the radiographer, nurse, or the like from suddenly becoming aware that mobile radiography can no longer be continued during the traveling diagnosis and becoming unable to do work in a scheduled manner, which avoids inefficient work. As a result, an efficient radiography schedule that enables the radiographer, nurse, or the like to do work in a scheduled manner is provided.

For example, the controller 101 generates a radiography schedule in which a pause process is interposed between dynamic radiography for the first human subject and dynamic radiography for the second human subject, which enables charging of the batteries, cooling of the radiation source 2, transmission of image data, and the like to be performed at a necessary time between radiography sessions when human subjects are changed during the traveling diagnosis.

For example, the controller 101 generates a radiography schedule in which a pause process is interposed between dynamic radiography in the first room and dynamic radiography in the second room, which enables charging of the batteries, cooling of the radiation source 2, transmission of image data, and the like to be performed utilizing a travel time during the traveling diagnosis.

For example, the controller 101 determines the length of the pause process based on information concerning the use environment, which enables a radiography schedule having a pause process of a length adapted to the use environment of the mobile radiography system 100 including the room temperature, the communication environment, and the like to be provided, for example.

For example, the controller 101 generates a radiography schedule including a first radiography schedule including a first pause process and a second radiography schedule including a larger number of pause processes than in the first radiography schedule, the pause processes each being shorter than the first pause process, which enables a radiography schedule that fits user's needs to be provided.

For example, the controller 101 generates a third radiography schedule not including a pause process, which enables a radiography schedule that fits user's needs to be provided in a case where the user does not intend to interpose a pause process.

For example, the controller 101 further generates a still radiography schedule through use of radiation, which enables an efficient radiography schedule to be provided also for still radiography in a traveling diagnosis.

The description in the above-described embodiment is a suitable example of the present invention, and is not a limitation.

In the above-described embodiment, the FPD cassette 3 is charged via the connector 108 when the FPD cassette 3 is placed in the housing 10, but this is not a limitation, and the FPD cassette 3 may be charged via a cable or the like.

The above-described embodiment has been described using, as an example, a case in which the display 103 serves as an output unit and a radiography schedule is displayed on the display 103, but, for example, the communicator 105 may serve as the output unit, and a radiography schedule may be output to an external device (such as, for example, a mobile terminal of a radiographer) via the communicator 105 to cause the external device to display the radiography schedule. Alternatively, the mobile radiography system 100 may include a printer, and a radiography schedule may be output to a sheet of paper by the printer.

Although the above description discloses an example of using a hard disk, a semiconductor nonvolatile memory, or the like as a computer-readable medium for the program according to the present invention, but this example is not a limitation. A portable recording medium such as a CD-ROM is applicable as another computer-readable medium. Carrier waves are also applied as a medium that provides data about the program according to the present invention via a communication network.

In addition, detailed configuration and detailed operation of each part constituting the mobile radiography system can also be changed within the scope of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese patent application No. 2021-002508, filed on Jan. 12, 2021, is incorporated herein by reference in its entirety.

What is claimed is:

1. A mobile radiography system that generates a radiography schedule of dynamic radiography through use of radiation, and performs the dynamic radiography, comprising:
an acquisition unit that acquires order information about each human subject concerning the dynamic radiography;
a generator that generates the radiography schedule of the dynamic radiography including a pause process based on the order information; and
an output unit that outputs the radiography schedule.

2. The mobile radiography system according to claim 1, wherein the generator interposes the pause process between dynamic radiography for a first human subject and dynamic radiography for a second human subject.

3. The mobile radiography system according to claim 1, wherein the generator interposes the pause process between dynamic radiography in a first room and dynamic radiography in a second room.

4. The mobile radiography system according to claim 1, wherein the generator determines a length of the pause process based on at least one of:
information concerning heat generation of a radiation source;
information about power storage concerning mobile radiography;
information about a memory that stores a dynamic image obtained by the dynamic radiography; and
information about wireless communication.

5. The mobile radiography system according to claim 4, wherein the information concerning heat generation of the radiation source includes at least one of:
information about a heat capacity of the radiation source; and
information about a temperature of heat generated by a single standard dynamic radiography session.

6. The mobile radiography system according to claim 4, wherein the information about power storage concerning mobile radiography includes information of at least one of:
a power storage capacity and a present amount of stored power of a radiography device; and
a capacity and a present amount of stored power of a power storage of a medical vehicle main body.

7. The mobile radiography system according to claim 4, wherein the information about the memory that stores the dynamic image obtained by the dynamic radiography includes information about a storage capacity and a present remaining capacity of an image storage of a medical vehicle main body.

8. The mobile radiography system according to claim 4, wherein the information about wireless communication includes:
information indicating a second position at which a radio wave is weaker than at a first position in a hospital; or
information about a communication speed at a predetermined position in the hospital.

9. The mobile radiography system according to claim 1, wherein the generator determines a length of the pause process based on information concerning a use environment.

10. The mobile radiography system according to claim 1, wherein the generator updates the radiography schedule based on the radiography schedule and an execution status of radiography.

11. The mobile radiography system according to claim 1, wherein the radiography schedule includes:
a first radiography schedule including a first pause process; and
a second radiography schedule having a larger number of pause processes than in the first radiography schedule, the pause processes each being shorter than the first pause process.

12. The mobile radiography system according to claim 1, wherein the generator generates a third radiography schedule of the dynamic radiography not including a pause process.

13. The mobile radiography system according to claim 1, wherein the generator generates a radiography schedule of a still image through use of radiation.

14. The mobile radiography system according to claim 1, further comprising a radiation source that emits the radiation.

15. The mobile radiography system according to claim 14, further comprising:
a radiography device that generates a radiation image based on the radiation emitted from the radiation source.

16. The mobile radiography system according to claim 15, further comprising:
a power storage that supplies power to the radiography device.

17. The mobile radiography system according to claim 16, wherein the power storage supplies power further to the radiation source.

18. A non-transitory computer-readable recording medium storing a computer-readable program that causes a computer that generates a radiography schedule of dynamic radiography through use of radiation to function as:
an acquisition unit that acquires order information about each human subject concerning the dynamic radiography;
a generator that generates the radiography schedule of the dynamic radiography including a pause process based on the order information; and
an output unit that outputs the radiography schedule.

19. A radiography schedule generation method of generating a radiography schedule of dynamic radiography through use of radiation, comprising:
acquiring order information about each human subject concerning the dynamic radiography;
generating the radiography schedule of the dynamic radiography including a pause process based on the order information; and
outputting the radiography schedule.
